(19) United States
(12) Patent Application Publication
(10) Pub. No.: US 2024/0115817 A1
(43) Pub. Date: Apr. 11, 2024

(54) CYCLONIC APPARATUS AND METHOD

(71) Applicant: Cambridge Healthcare Innovations Limited, Cambridge Cambridgeshire (GB)

(72) Inventor: David HARRIS, Cambridge (GB)

(21) Appl. No.: 18/546,170

(22) PCT Filed: Feb. 11, 2022
(86) PCT No.: PCT/GB2022/050381

§ 371 Date: Aug. 11, 2023

(30) Foreign Application Priority Data
Feb. 12, 2021 (GB) 2102026.8

(51) Int. Cl. A61M 15/00

(52) U.S. Cl. CPC A61M 15/0095; A61M 15/0021; A61M 15/0086; A61M 2202/064; A61M 2206/16; A61M 2206/20

(57) ABSTRACT

A cyclonic apparatus having an inlet and an outlet comprises a first cyclone chamber having a first cyclone inlet, a first reduced-pressure inlet forming or in fluid connection with the cyclonic-apparatus inlet, and a first outlet forming the cyclonic-apparatus outlet. The first cyclone chamber is operable to establish a cyclonic flow of fluid between the first cyclone inlet and the first outlet in response to fluid being drawn from the first outlet, so as to create a first reduced-pressure zone of fluid at the first reduced-pressure inlet. A drug delivery device in which a source of a medicament is coupled to the cyclonic-apparatus inlet of a cyclonic apparatus.

This invention relates to a cyclonic apparatus for reducing fluid (gas) pressure, and an inhaler such as a dry-powder inhaler having or comprising the cyclonic apparatus. In particular, this may be a medical apparatus and method.

### BACKGROUND

All current dry powder inhalers (DPIs) rely solely upon harnessing a proportion of the available energy from the patient's inhalation to do work on the powdered formulation, to break it up (deagglomerate the particles), to produce a fine, respirable aerosol. The vast majority of DPIs deliver formulations that comprise two or more different particle size fractions—the “fine” or “respirable” fraction is the drug, also known as the Active Pharmaceutical Ingredient (API), and the bulk of the formulation is the “carrier” fraction, which is comprised of much larger (coarse) non-respirable particles—usually lactose. This is done for two main reasons:
i) Many drugs that are delivered by inhalation are potent, and only require typically 50 μg to a few hundred μg per dose. This is volumetrically a very small amount, and consequently difficult to meter—either in the factory by specialist filling equipment, or inside the inhaler. Mixing this tiny quantity of API with a much larger quantity of inert and coarse carrier particles, typically lactose, bulks up the volume to be metered and therefore increases both the accuracy and consistency of dose metering;
ii) Respirable particles typically need to be less than 5 μm in aerodynamic diameter to travel down into the lungs and not impact earlier on, upstream in the larger bronchioles. Many therapies require deep lung deposition—some as far as the alveoli—and consequently require even finer respirable particles, often referred to as “extra-fine”, which are typically under 2 μm in aerodynamic diameter. All fine particles, such as these, do not “flow” well, meaning handling them during manufacture is very difficult. A simple example would be to compare icing sugar with granulated sugar—identical material, but with different size fractions. If you try to pour icing sugar from a spoon, it doesn't flow, as the average particle size is only a few tens of micrometers. Conversely, granulated sugar, which has an average particle size of a few hundred micrometers, pours from a spoon very easily. By mixing fine, respirable API with a much coarser carrier fraction of lactose to improve the overall flowability, handling the formulation during manufacture becomes much more straightforward.

However, a well-mixed (homogenous) formulation comprising one or more fine APIs with a coarse carrier fraction is difficult to deagglomerate and aerosolise. Many market leading DPIs only achieve 20 to 30% Fine Particle Fraction (FPF), which is the percentage of drug (of the dose emitted from the inhaler) that is below 5μm—also known as the “respirable fraction”. The remaining drug is predominantly still attached to the carrier particles, which are usually between 50 μm and 200 μm—i.e. much larger than the API particles. It is worth noting that a typical API particle might be 2 μm in diameter, whereas a typical carrier particle size, e.g. DFE Pharma's Lactohale® 200, is approximately 80 μm. Whilst this difference is only a factor of ˜40× in diameter, it is a difference of approximately 60,000× in terms of mass. The physics governing the behaviour of API particles is very different to that of carrier particles, and is indeed complex for both.

As a patient inhales—from any current DPI that uses a carrier-based formulation—virtually all of the formulation leaves the inhaler and enters the patient, except for a small percentage that may be held up within the inhaler. (It is certainly the design intent of DPIs to deliver the entire dose to the patient, and ideally have no formulation—API or carrier fraction—remaining within the inhaler device). As the formulation travels with the airflow into the patient's mouth, the carrier particles have sufficiently high inertia such that they are unable to turn with the airflow towards the back of the patient's mouth, and travel down with the airflow into the trachea. Because they are aerodynamically so large, they instead impact and deposit upon the back of the patient's throat. In fact, most particles with an aerodynamic diameter of over 10 μm will not be able to follow the airflow into the trachea, due to their high inertia, and will consequently impact and deposit on the patient's throat. This means that for many inhalers that are available and used today, the majority of the API (drug) is delivered to the patient's mouth and throat, as it is still attached to the carrier particles, rather than reaching the target site of the lungs. For APIs that are steroids, for example, this topical delivery to the mouth and throat region is highly undesirable, and can lead to unwanted side effects such as candidiasis. This does very little to help the patient using the inhaler to remain compliant and adherent to their therapy. Previous inventions, such as the Conix® DPI (Patent application WO2006061637), have sought to retain the vast majority of the lactose carrier fraction in the inhaler to minimise mouth and throat deposition. The Occoris® DPI (Patent application WO2015082895) is designed to work with fine API particles only, and requires zero carrier particles, thus greatly reducing any mouth and throat deposition, as all the emitted aerosol is sufficiently fine and has sufficiently low inertia, such that it is able to follow the airflow into the trachea and avoid impacting on the back of the patient's throat. However, with both of these inventions, and similar, the patient using them receives negligible feedback in the form of taste, that the inhaler has delivered a dose. This is potentially problematic and even dangerous; as the patient could think that their inhaler has not delivered a dose, and may repeat the inhalation one or more times and risk the possibility of overdosing.

Most DPIs emit a dose that comprises almost all of the API and almost all of the carrier fraction. The entrainment of the dry powder formulation happens very quickly, in the first part of the inhalation (inspiratory manoeuvre), and often all of the powder has left the inhaler within the first few hundred milliseconds. As DPIs by their very nature have resistance to the airflow (airflow resistance), a full inspiratory manoeuvre may last several seconds. One advantage of retaining the carrier fraction within the DPI deagglomeration engine (a reverse-flow, frusto conical cyclone, in the case of the Conix DPI), is that work is done on the formulation to detach and deagglomerate the fine API from the carrier particles throughout the entire duration of the inspiratory manoeuvre. As this time period during which work is done on the formulation is now several seconds rather than a few hundred milliseconds, much more thorough deagglomeration can be achieved, which results in a higher FPF—i.e. more drug reaches the deep lungs of the patient, and less remains to be deposited in the mouth and throat region.

Currently DPIs either emit almost everything, or retain virtually the entire carrier fraction and only emit the fine, aerosolised API (in the case of Conix). What would be advantageous would be a deagglomeration system in which the emission rate and quantity of the carrier fraction could be tuned, to efficiently balance the quantity of emitted carrier fraction required to produce useful (taste) feedback to the user, versus the quantity retained within the deagglomeration system to do sufficient work on the formulation to produce a highly efficient FPF.

A huge challenge for all current DPIs is that because they are solely reliant upon harnessing energy from the patient's inspiratory manoeuvre, it is very difficult to achieve consistent delivery of drug when the energy available varies considerably from patient to patient. All currently available DPIs are “passive”, in that they do not have their own energy source, unlike pressurised metered dose inhalers (pMDIs), which contain hydrofluoroalkane (HFA) propellant to produce a droplet aerosol by flash evaporation through a spray orifice (similar to hairspray or other spray cans, only metered). Whilst “active” DPIs are, and have been, in development, right now there are none commercially available. Active DPIs, such as Occoris, overcome the huge variability from one user to another by containing an internal energy source to produce a respirable aerosol, which is independent of the user, or more specifically, it is independent of how the user inhales. There are probably no active DPIs available because they are so complicated to design, optimise and produce. The ideal DPI system comprising a deagglomeration engine, or deagglomeration apparatus, is a system that consistently produces a high fine particle fraction, independently of how the user inhales, and is simple and cost effective to manufacture. It is also small in size and a platform technology that can be used equally across a range of different inhaler types. For example, it could be incorporated into a single-use inhaler, that is discarded after the delivery of one single dose, e.g. for the delivery of vaccines. It could be incorporated into a single-dose reusable inhaler, e.g. for the delivery of non-routine therapies such as pain relief, insulin for diabetes management, etc. It could also be incorporated into a multi unit-dose inhaler, which may contain 30 to 60 individual, pre-metered doses, and is suitable for delivering routine maintenance medication for the treatment of asthma and COPD, on a once or twice daily regimen over a period of a month, for example. The advantage of using a core engine that is a platform across different device embodiments is that the performance remains identical across them all.

There are therefore a number of problems in the prior art relating to DPIs, including; to provide a simple, low-cost core platform technology that is suitable for incorporation into a wide range of dry powder inhalers (DPIs) and is suitable for incorporating an adaptive classification system that:
i) Produces a high and consistent fine particle fraction (FPF) which is independent of how strongly the user inhales; and
ii) Enables the rate of carrier fraction emission from the formulation unit container (e.g. a coldform/foil blister) to be easily tuned.

To improve on conventional DPIs, the primary aim of this adaptive classification system would be to emit only fully deagglomerated, fine API particles. A secondary aim would be that any coarse lactose (or other) carrier particles which might be emitted will ideally have been completely stripped of API particles. This may be achievable by tuning the system to delay the emission of carrier particles towards the end of the inspiratory manoeuvre—such emission being governed by an influenceable probabilistic function. Delaying carrier particle emission facilitates more complete deagglomeration, by extending the time over which work can be done on the formulation, resulting in higher and more consistent FPF. This also means that any carrier particles emitted are more likely to be free of API particles, thus minimising API deposition in the mouth and throat. It further means that the patient may only tend to taste the carrier particles towards the end of the inspiratory manoeuvre, which may make this an effective indication that dose delivery is complete.

### Background Information on Inspiratory Energy

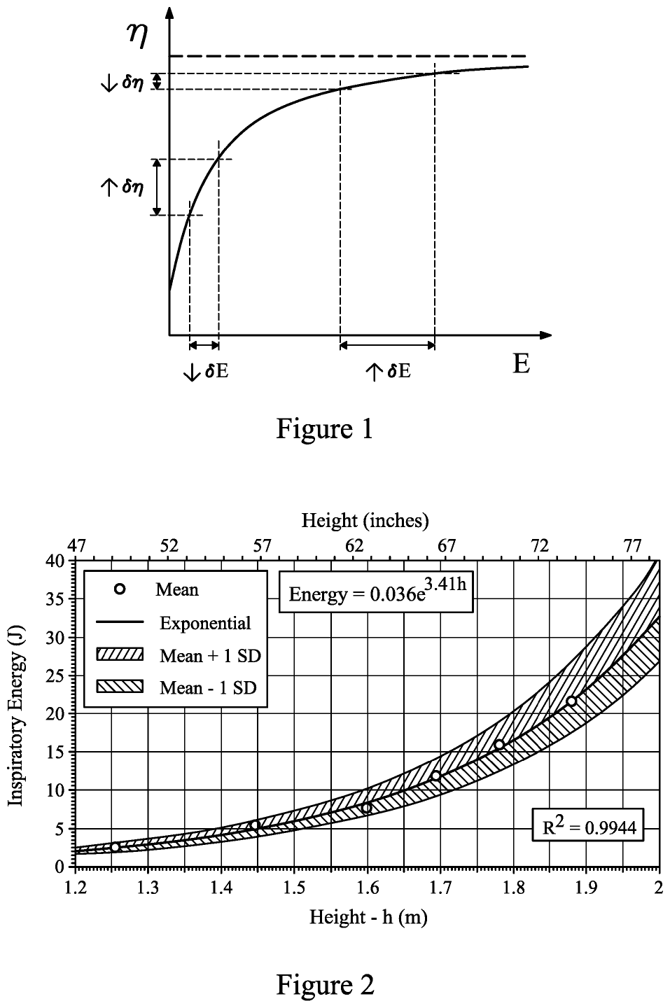

If you examine the relationship between the energy put into a formulation (powder) during inhalation and the efficiency of a deagglomeration engine—i.e. energy harnessed from the inspiratory manoeuvre of the patient and input to the deagglomeration and aerosolisation of the formulation, and the effectiveness of that deagglomeration and aerosolisation of the formulation—there are a few key observations to note, FIG. 1 illustrates the effect of input energy (energy applied to the powder) (x-axis) on the aerosolisation (fine particle) efficiency (y-axis) of typical passive DPIs. Firstly, with almost zero input energy, the most loosely bound API particles will readily detach from their carrier particles simply by being entrained in the airflow, giving rise to the “free” part of the fine particle fraction. The earliest DPIs, such as the Aerohalor® (Abbott Laboratories, 1947), transferred very little energy into the powdered formulation, and consequently only achieved this “free” part of the fine particle fraction—of the order 10-15%, indicated as the intersection with the y-axis, FIG. 1.

Secondly, with current DPIs (e.g. Turbuhaler®, of AstraZeneca, NEXThaler® of Chiesi, Genuiair® of AstraZeneca, Onbrez®, of Novartis, etc.) a higher proportion of the energy available from the patient's inspiratory manoeuvre is transferred to the formulation, which results in greater efficiency and a higher fine particle fraction, FIG. 1. However, it is important to note that, using airflow alone, it is impossible to ever achieve 100% efficiency, as the most tightly bound API particles that are directly on the surface of carrier particles, or even mechanically locked in place in microscopic cracks or imperfections on the carrier particle's surface, cannot be removed without breaking down the close range forces of adhesion—e.g. they would need to be washed off with liquid or dissolved solvent. Aerodynamic deagglomeration is a probabilistic system—every time a carrier particle collides with another, or with a wall within the deagglomeration engine, for example, there is a chance that API particles will become detached. By the very nature of a probabilistic system, achieving a 100% is infinitely unlikely, hence the efficiency curve asymptotes towards 100%, FIG. 1.

This efficiency curve can be considered as two parts—a steep part and a flatter part. All current DPIs perform (with typical carrier based formulations, at least) on the steep part of this curve, and the “knee” also known as the point where the curve visibly bends, specifically from the steep part to the flatter part, is located at approximately 60-70%. This is a particularly suboptimal operating region, as even a small difference in the input energy results in a large difference in the efficiency. This is evident in real use with the majority of DPIs: Patients who inhale less forcefully put less energy into the formulation and consequently receive a lower fine particle dose than patients who inhale strongly, who put greater energy into the formulation and receive a higher fine particle dose. As one of the primary aims of any respiratory drug delivery system is to deliver a known quantity of drug, independently of how the patient inhales—operating before the knee, on the steepest part of the Energy-Efficiency curve—is unfortunately the worst place to be. It is one of the main reasons why so few DPIs make it to market, as they struggle to make it through clinical studies due to variability in delivered dose, resulting from the wide range of input energy provided by the patient group in the study. It has been shown that the total available inspiratory energy correlates well with a patient's height, irrespective of age or gender, FIG. 2 (Harris, D. S., Scott, N., Willoughby, A., How does airflow resistance affect inspiratory characteristics as a child grows into an adult?, DDL21 Conference Proceedings, 79-87 (December 2010)).

The data shown in FIG. 2 are for healthy volunteers, and yet there is still a ten-fold difference in the average inspiratory energy within the ˜90 volunteers, ranging from ˜3 J to over 30 J. Patients with severe asthma or late stage COPD will extend this range of inspiratory energy considerably. This is a key reason why designing DPIs, that are powered by the patient's lungs, is so challenging, particularly achieving consistent delivery efficiency with such a broad range of input energy.

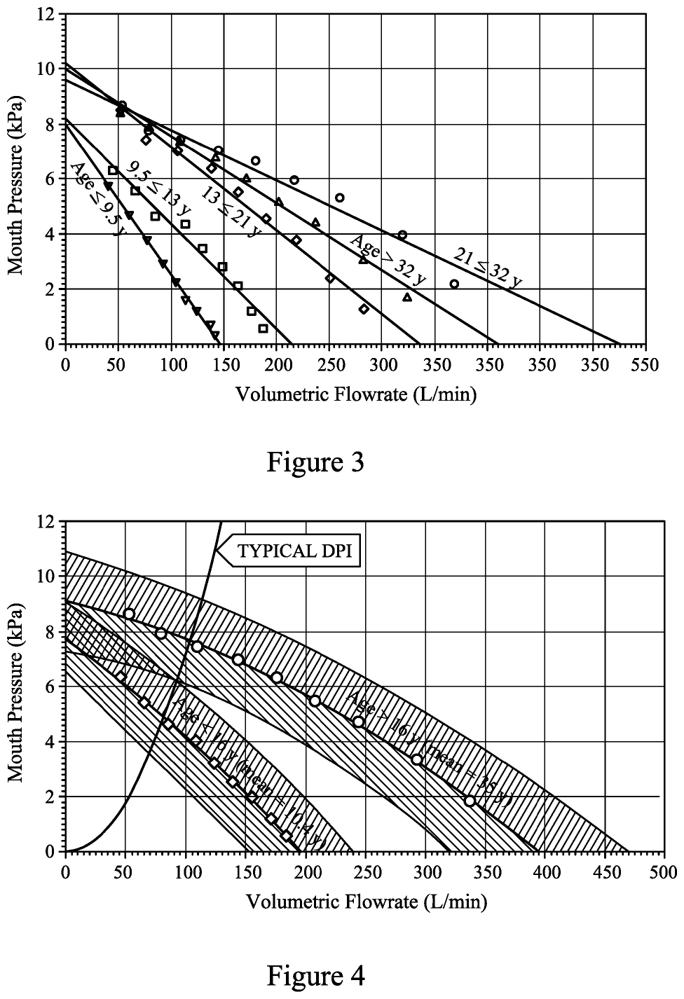

Looking at the Flow-Pressure relationship across range of airflow resistances within this same study, it is clear that there is much greater variation in peak inspiratory flowrate (PIFR) than there is in Mouth Pressure, FIG. 3 (Harris, D. S., Scott, N., Willoughby, A., How does airflow resistance affect inspiratory characteristics as a child grows into an adult?, DDL21 Conference Proceedings, 79-87 (December 2010)). Again, this will be exacerbated with the inclusion of asthmatic and COPD patients. What these data do suggest is that it is advantageous to design higher resistance DPIs, as they will operate closer towards the left of the graph in FIG. 3, where there is greater consistency between all users. This is illustrated in FIG. 4 (Harris, D. S., Scott, N., Willoughby, A., How does airflow resistance affect inspiratory characteristics as a child grows into an adult?, DDL21 Conference Proceedings, 79-87 (December 2010)).

Another important observation from these results is that healthy volunteers are likely to create a pressure drop across a medium resistance DPI of approximately 4 kPa to 8 kPa. If, however, the resistance of the DPI were higher, it would be reasonable to expect a smaller pressure range, at higher pressures, of approximately 6 kPa to 9 kPa.

### SUMMARY OF INVENTION

The invention provides a cyclonic apparatus and a drug-delivery device as defined in the appended independent claims, to which reference should now be made. Preferred or advantageous features of the invention are set out in dependent subclaims.

The cyclonic apparatus may advantageously transform the energy available from a patient's inspiratory manoeuvre, which is typically high flowrate and low magnitude negative pressure, into a much higher magnitude negative pressure, albeit at a much lower flowrate.

This is highly advantageous, as typical quantities of formulation (powder) in DPIs are very small—usually up to 10 mg per dose. Most conventional DPIs have quite low airflow resistance, which results in people inhaling through them at 30 to 150 LPM (litres per minute)—depending on the inhaler. Even with the highest resistance DPIs, such as Boehringer Ingelheim's HandiHaler®, many users will inhale at least 30 LPM. A litre of air weighs ˜1.2 g (at standard temperature and pressure), so even at the lowest flowrates achieved through current DPIs, this equates to ˜0.5 L/s, or 600 mg/s in terms of the mass flowrate. The point to note is that even this low airflow rate is considerably more massive than the quantity of API (and carrier) that needs to be deagglomerated and aerosolised. The invention may enable trading this unnecessarily high flowrate for an increase in the magnitude of negative pressure. In this way, it may be possible to achieve much higher performance by increasing the effectiveness of the energy transfer into the powder formulation. This is because the airflow velocities that can be reached (e.g. within a deagglomeration engine) directly result from the pressure drop achieved, in accordance with Bernoulli. Moreover, the kinetic energy of the airflow is proportional to the square of the airflow velocity—so doubling the airflow velocity results in four times the kinetic energy. This is important for any DPI design, as it is the kinetic energy available in the airflow that may do work on the dry powder formulation in order to deagglomerate the particles and create a fine, respirable aerosol.

A further advantage of embodiments of the invention may be to transform and normalise the (variable) input energy available from different users, so that the energy used to deagglomerate and aerosolise the powdered formulation remains more consistent between different users or patients, even if they are capable of different inhalation pressures and air flow rates. This is explained in more detail later on in this document.

Embodiments of the invention use the principle of swirling flow to effectively amplify (negative) pressure by trading a reduction in flowrate. The simplest definition of a cyclone is a fluid rotating around a low pressure core. In a well designed swirl chamber (cyclone chamber) (i.e. not necessarily only reverse-flow cyclones—this is equally applicable to uniflow/through-flow cyclones, in which the air flows into one end and exits at the other), the core pressure can be considerably lower than the driving pressure. For example, it is quite reasonable to achieve a core pressure that is 1.6× that of the driving pressure—i.e. in an inhaler if the patient inhales through a swirl chamber at a (mouth) pressure drop of −4 kPa, then the pressure in the core of the swirling vortex could in a preferred embodiment be 1.6×−4=−6.4 kPa. Whilst it is possible to achieve higher amplifications of core pressure, a reasonably conservative factor of 1.6× is used in as a preferred illustrative embodiment in this disclosure.

In a uniflowfrusto-conical swirl chamber, one or more tangential inlets create a swirling flow within the chamber, and due to conservation of angular momentum, the tangential velocity of the swirling airflow increases as the effective chamber diameter reduces, due to the conical nature of the geometry. This is the basis of all conical cyclones—e.g. as used in bagless vacuum cleaners—the high centripetal acceleration created within the cyclone is able to separate small dust particles by overcoming the aerodynamic drag force exerted on them—a cyclonic separator. In a uniflow design, however, all the particles are emitted rather than collected.

Thus, in a first aspect the invention may provide a cyclonic apparatus for reducing fluid pressure. The apparatus may comprise a cyclone means for reducing the pressure of a fluid, passing therethrough, optionally in a stepwise fashion. Preferably the cyclone means may comprise a first cyclone having a fluid outlet and a fluid inlet. The first cyclone may be operable to establish a cyclonic flow of fluid from the inlet to the outlet, preferably so as to create a first reduced pressure zone of fluid at the inlet. In one embodiment, the cyclonic apparatus, or cyclone means, may comprise only one cyclone, the first cyclone. But in a further embodiment the cyclone means may further comprise a second cyclone having a fluid outlet in the first reduced pressure zone and a fluid inlet. The second cyclone may achieve a further reduction in fluid pressure so as to create a second reduced pressure zone of fluid at a pressure lower than that of fluid in the first zone.

The second cyclone may be so arranged relative to the first cyclone that, in use, the second cyclone continues the cyclonic flow established by the first cyclone.

The cyclone means may be unidirectional.

Each of the first and second cyclone may comprise a respective conduit having an inlet and an outlet and being tapered from inlet and to outlet end.

The conduits may be coaxial and the outlet end of the second conduit may be nested within the first conduit.

The first cyclone may have at least one further inlet at a position spaced from an axis of the cyclonic flow established by the first cyclone.

The second conduit may be axially spaced from the first conduit, so as to define the further inlet.

The cyclone means may comprise a third cyclone having a conduit which is tapered from an inlet end to an outlet end, and which may be partially nested within the conduit of the second cyclone. The conduits of the second and third cyclone may be spaced from each other to define a further, non-axial inlet for the second cyclone.

In a second aspect the invention may provide a dry powder inhaler into which a dose of medicament having an active pharmaceutical component can be loaded. The dry powder inhaler may have a cyclonic apparatus in accordance with the apparatus as described. The dry powder inhaler may further comprise a mouthpiece which is in fluid communication with the outlet of the first cyclone. In use, the cyclonic apparatus may amplify the pressure reduction, caused by a user inhaling through the mouthpiece, and apply the amplified reduced pressure to the dose, for example in a deagglomerator, to release the active pharmaceutical component and enable that component to be inhaled through the mouthpiece.

### PREFERRED EMBODIMENTS OF THE INVENTION

Preferred embodiments of the invention will now be described by way of example, with reference to the accompanying drawings, in which:

FIG. 1: A graph illustrating the effect of input energy (energy applied to a powder) on aerosolisation (fine particle) efficiency of typical passive DPIs;

FIG. 2: A graph illustrating the relationship between inspiratory energy and height;

FIG. 3: A graph illustrating the effect of age upon pressure and flowrate;

FIG. 4: A graph showing children and adults' Mouth Pressure;

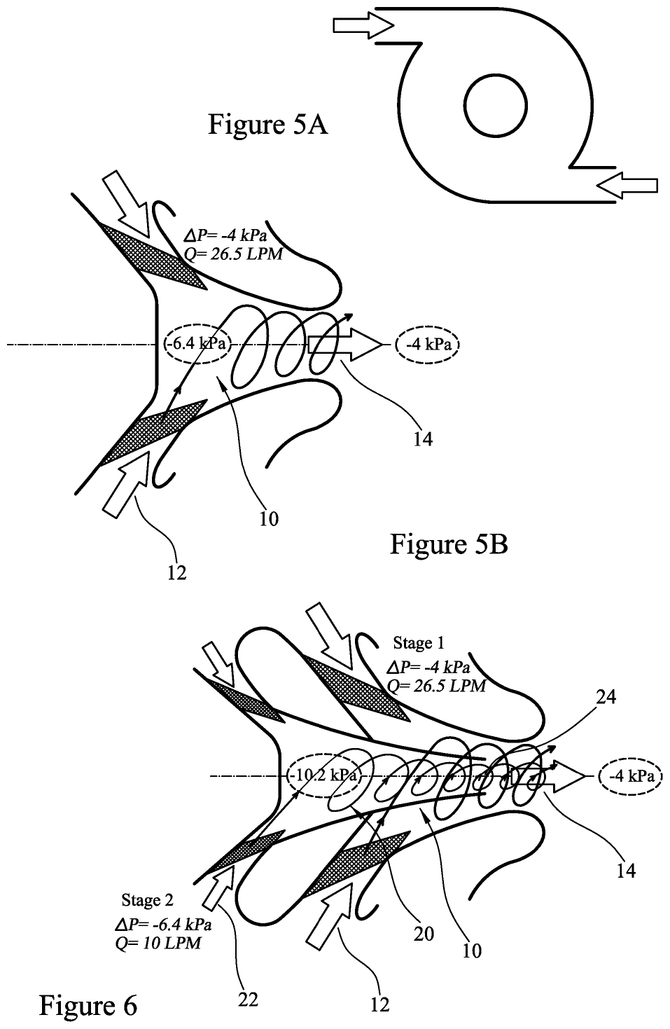

FIG. 5A: End view of uniflow frusto-conical, twin-inlet swirl chamber embodying the invention;

FIG. 5B: Side view of uniflow frusto-conical, twin-inlet swirl chamber of FIG. 5B;

FIG. 6: Side view of two-stage nested swirl chamber embodying the invention, showing previous core pressure produced by Stage 1 being used to drive Stage 2;

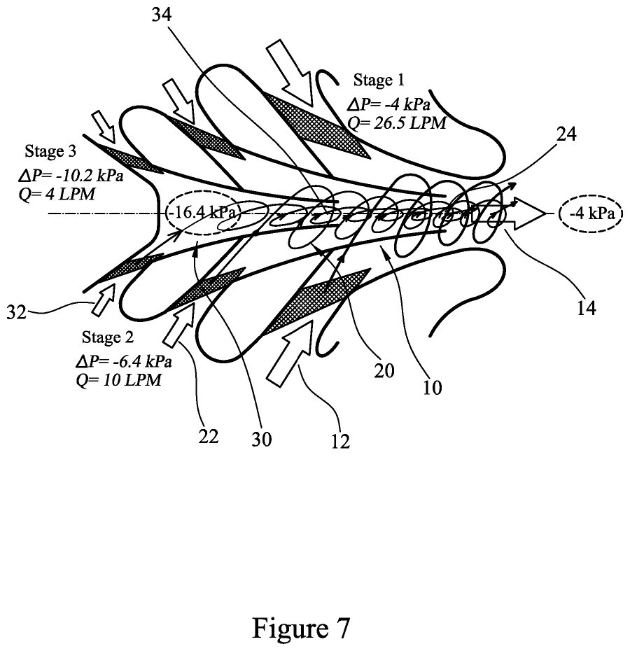

FIG. 7: Side view of three-stage nested swirl chambers embodying the invention, to achieve greater amplification of pressure;

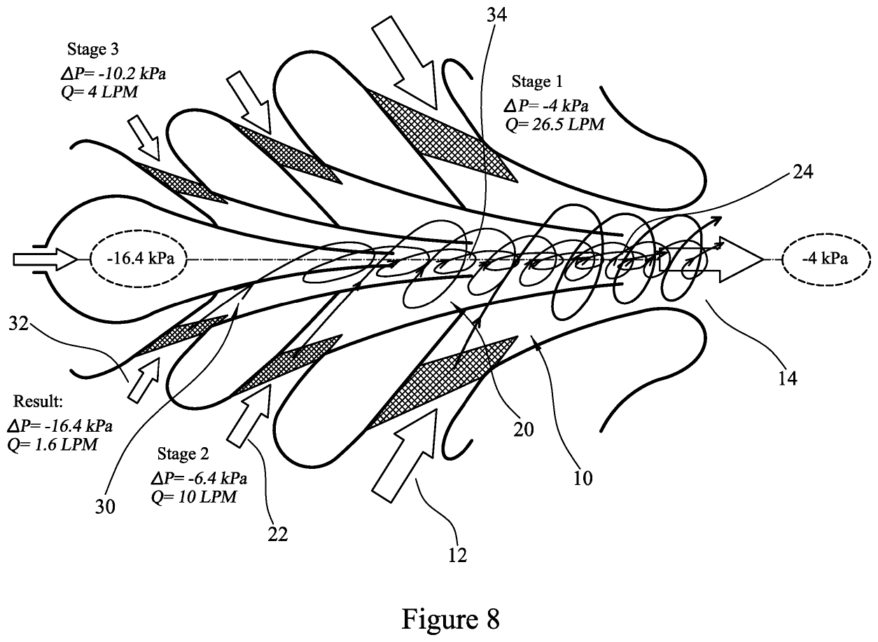

FIG. 8: Diagram of whole (three-stage) amplification system embodying the invention; and

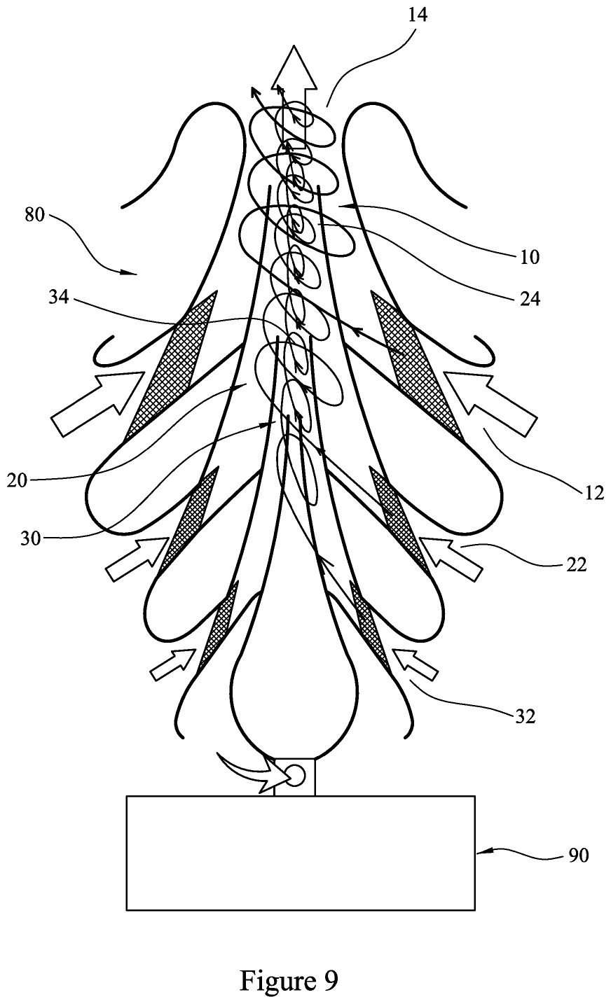

FIG. 9: Diagram of the cyclonic apparatus amplification system in a system with a classification deagglomeration engine embodying the invention.

If a cyclonic apparatus has a conical swirl chamber geometry that is designed so that a pressure drop across it of −4 kPa creates a flowrate through it of 26.5 LPM, then using the 1.6× amplification factor discussed earlier, a maximum (negative) core pressure of −6.4 kPa can be achieved, FIGS. 5A and 5B.

Let's now consider this first uniflow swirl chamber 10 as “Stage 1”, and use the newly amplified core pressure to drive a second, smaller stage, Stage 2 20, as shown in FIG. 6. Stage 2 20 is a smaller swirl chamber that is designed to run at a flowrate of 10 LPM with a driving pressure of −6.4 kPa. When combined with Stage 1, in a nested and coaxial manner, the core pressure of Stage 1 10 drives the smaller Stage 2 20 at a flowrate of 10 LPM. Using the pressure amplification factor of 1.6× as before, the new core pressure is now −10.2 kPa, and the total (combined) flowrate through Stages 1 10 and 2 20 is 36.5 LPM, FIG. 6.

It is important to note that, instead of a second, smaller cyclonic amplification stage, the amplified core pressure of the first stage could be used to directly drive an inhaler deagglomeration engine, for example, albeit at a more moderate level of amplification. This could be achieved by creating an additional (axial) inlet at the back of the Stage 1 cyclone 10 shown in FIGS. 5A and 5B such that the amplified core pressure of −6.4 kPa can now drive the inhaler deagglomeration engine (or anything else) at 1.6× the main driving pressure—i.e. the deagglomeration engine could be connected in the same manner as a second cyclonic amplification stage, as shown in FIG. 6. The potential advantages of only having a single stage cyclonic amplifier are that; i) less flowrate is traded therefore higher flowrate is available to power the deagglomeration engine, which may be particularly advantageous for high dose masses and, ii) the manufacturing and fabrication of a single stage amplifier is likely to be simpler and cheaper than a more complex multi-stage amplifier.

Of course further stages can be added to continue to amplify the driving pressure, using the same principle—albeit the flowrate through each additional stage must reduce each time another is added, FIG. 7. It is worth noting that allowing too much additional axial flow (i.e. not through the tangential inlets) will prevent the optimum development of the swirl, and reduce the overall pressure amplification. In this example configuration, a conservative figure of <0.4× has been used. FIG. 7 shows a three-stage cyclonic amplifier that results in a total pressure amplification of just over 4×.

So if the core of swirl developed in Stage 3 30 is tapped into to drive a deagglomeration engine, it is possible to drive this engine at −16.4 kPa with a flowrate of 1.6 LPM, FIG. 8. The total flowrate through the system is now ˜42 LPM (26.5+10+4+1.6), for a total pressure drop across it of −4 kPa. For the user, this feels like a typical “high” resistance DPI—such as HandiHaler, for example. However, a deagglomeration engine for use with the cyclone amplifier can be designed to have a flowrate through it of just 1.6 LPM but at a pressure drop of −16.4 kPa—i.e. over four times the mouth pressure provided by the patient inhaling. As the maximum airflow velocity is proportional to the square-root of the driving pressure (for turbulent flow), then the peak airflow velocity within the deagglomeration engine will be double the maximum possible value that could be achieved without the amplification system. However, the kinetic energy is proportional to the velocity squared multiplied by the mass of air, so without the reduction in mass flowrate would therefore be four times greater than with no amplification, i.e. proportional to the increase in driving pressure. However, energy cannot be created, and the trade-off in this system is flowrate—the mass flowrate of the air flowing through the deagglomeration engine will be a fraction of the airflow rate through the system as a whole—just 1.6 LPM compared to 42 LPM. The isentropic power of the deagglomeration engine is only 1.6/60 (LPS)×16.4 kPa=0.43 airwatts (aw); whereas the isentropic power for the entire system is 42/60 (LPS)×4 kPa=2.8 aw—approximately 6.5× higher. This is an interesting and perhaps counterintuitive observation to note—we have reduced the kinetic energy available within the deagglomeration engine by a factor of −6.5×, yet this energy is more suitable to deagglomerate the small quantity of powdered formulation than the original energy, without the pressure amplification system. An analogy would be using hammers to crack nuts. If you take a sledge hammer on a pendulum, and swing it such that it doesn't quite have enough kinetic energy to crack a nut (which is up against a rigid stop), and then change it for a pin hammer with exactly the same kinetic energy, the pin hammer will smash the nut to pieces. This sledge hammer/pin hammer analogy has been used to explain why patients do not provide the ideal type of energy to deagglomerate powdered formulations effectively.

One very important point to note is that in the hammer analogy, all of the kinetic energy of the hammer is absorbed or dissipated by the nut—this is different to the transfer of energy from airflow to powder in deagglomeration engines. In a typical DPI, the majority of the airflow simply passes by the formulation; the air follows the path of least resistance. So if you have, for example, a bulk of powder resting in a dose container (cavity), most of the air will simply flow over the powder without imparting any energy into it—i.e. most of the energy available in typical DPIs is wasted. Eventually, the powder will become entrained into the airflow, acquire momentum, and either travel directly out of the device, or more advantageously will impact against walls or other particles, as this impact and sudden exchange of momentum is the most effective way to detach and release particles from one another. Swirl chambers are commonly used to promote the frequency of particle-wall and particle-particle impacts, as the carrier particles travel through the swirl chamber in a helical path, at or close to the wall. This is because the centripetal force experienced by the relatively large carrier particles easily overcomes the aerodynamic drag force pulling them inwards towards the axis of rotation—therefore they are swung outwards and travel along the inner wall of the swirl chamber, colliding with asperities on the wall and other particles, and each time there is an impact, there is a chance that API particles attached to them will become detached, entrained in the airflow, and effectively aerosolised. This is the principal method of deagglomeration and aerosolisation within swirl chambers used by DPIs. A novel swirl chamber that is specifically designed to use the transformed kinetic energy provided by the pressure amplification system described above, can provide much more effective deagglomeration than classic swirl chambers that are powered directly by the inspiratory energy of the patient.

To summarise, a preferred embodiment of this invention may provide a cyclonic apparatus that is a pressure (vacuum) amplifier to amplify the (negative) mouth pressure produced by the patient when inhaling A system that uses this invention combined with a classification system designed specifically to run at much lower flowrates and much higher pressure drops than in typical dry powder inhalers would be particularly advantageous. Through the combination of the cyclonic apparatus that is a pressure (vacuum) amplifier and a classification and de-agglomeration system, turbulent flow regimes can be established in a (typically) small blister cavity, enabling the carrier particles to acquire sufficient inertia to recirculate within the cavity and thereby increase the time window to transfer kinetic energy from the airflow into the formulation, and achieve high fine particle efficiency.

FIG. 9 shows the cyclonic apparatus in a system with a deagglomeration engine 90.

The transformation of a patient's (typically high flowrate+low pressure drop) inspiratory energy into a more useful (low flowrate+high pressure drop) energy preferably enables the creation of an optimal flow regime within a classification deagglomeration engine, and consequently moves the performance into the flatter region at the right-hand side of the Energy—Efficiency curve (FIG. 1). Operating in this region of the Energy—Efficiency curve achieves two advantageous results:
i) The fine particle fraction is much higher, meaning more drug goes into the deep lung and less drug is deposited in the mouth and throat of the patient, and;
ii) As this part of the curve is flatter, any variation in the strength of the inspiratory manoeuvre between patients results in less variation in the delivered dose, meaning delivered dose uniformity is better.

By way of a summary, preferred embodiments and features of the invention are set out as a list of numbered clauses below.
1. A cyclonic apparatus having an inlet and an outlet 14 and comprising:
   a first cyclone chamber 10 having a first cyclone inlet 12, a first reduced-pressure inlet forming or in fluid connection with the cyclonic-apparatus inlet, and a first outlet 14 forming the cyclonic-apparatus outlet 14;
   the first cyclone chamber 10 being operable to establish a cyclonic flow of fluid between the first cyclone inlet 12 and the first outlet 14 in response to fluid being drawn from the first outlet, so as to create a first reduced-pressure zone of fluid at the first reduced-pressure inlet.
2. A cyclonic apparatus according to clause 1, comprising:
   one or more further cyclone chambers arranged in series upstream of the first cyclone chamber 10, each further cyclone chamber having a cyclone inlet, a reduced-pressure inlet, and an outlet coupled to the reduced-pressure inlet of the next-downstream cyclone chamber;
   each further cyclone chamber being operable to establish a cyclonic flow of fluid between its cyclone inlet and its outlet and to create a reduced-pressure zone of fluid at its inlet in response to fluid being drawn from its outlet by the reduced-pressure zone of fluid in the next-downstream cyclone chamber;
   in which the pressure in the reduced-pressure zone of each cyclone chamber is lower than the pressure in the reduced-pressure zone of the next-downstream cyclone chamber;
   and in which the reduced-pressure inlet of the cyclone at an upstream end of the series of cyclones forms the cyclonic-apparatus inlet.
3. A cyclonic apparatus according to clause 1, further comprising:
   a second cyclone chamber 20 having a second outlet 24 coupled to the first reduced-pressure inlet, a second cyclone inlet 22 and a second reduced-pressure inlet,
   the second chamber 20 being operable to create a second reduced-pressure zone of fluid in response to fluid being drawn from the second outlet 24 by the first reduced-pressure zone, wherein the fluid in the second reduced-pressure zone is at a lower pressure than the fluid in the first reduced-pressure zone.
4. A cyclonic apparatus according to clause 3, further comprising:
   a third cyclone chamber 30 having a third outlet 34 coupled to the second reduced-pressure inlet, a third cyclone inlet 32 and a third reduced-pressure inlet,
   the third chamber 30 being operable to create a third reduced-pressure zone of fluid in response to fluid being drawn from the third outlet 34 by the second reduced-pressure zone, wherein the fluid in the third reduced-pressure zone is at a lower pressure than the fluid in the second reduced-pressure zone.
5. A cyclonic apparatus according to any preceding clause, in which each cyclone chamber in the series progressively amplifies, at its reduced-pressure inlet, a reduced pressure applied, in use, to the cyclonic-apparatus outlet.
6. A cyclonic apparatus according to any preceding clause, in which each cyclone chamber is unidirectional.
7. A cyclonic apparatus according to any of clauses 2 to 6, in which the cyclones are arranged coaxially with each other.
8. A cyclonic apparatus according to any preceding clause, in which the first cyclone chamber comprises a conduit having an inlet end and an outlet end, and being tapered from a larger diameter at the inlet end to a smaller diameter at the outlet end.
9. A cyclonic apparatus according to any of clauses 2 to 8, in which each cyclone chamber comprises a conduit having an inlet end and an outlet end, and being tapered from a larger diameter at the inlet end to a smaller diameter at the outlet end.
10. A cyclonic apparatus according to clause 9, in which the conduits are coaxially arranged and in which the outlet end of the each conduit is nested or arranged within the inlet end of the next-downstream conduit.
11. A cyclonic apparatus according to any preceding clause, in which the cyclone inlet of the or each cyclone chamber is spaced along an axis of the cyclone formed, in use, in that cyclone chamber.
12. A cyclonic apparatus according to clause 11, in which the cyclone inlet of the or each cyclone chamber is tangential to the cyclone formed, in use, in that cyclone chamber.
13. A cyclonic apparatus according to any preceding clause, comprising two or more cyclone chambers arranged in series, in which each cyclone chamber has a higher airflow resistance than the next-downstream cyclone chamber.
14. A drug-delivery device 80 in which a source of a medicament 90 is coupled to the cyclonic-apparatus inlet of a cyclonic apparatus as defined in any preceding clause.
15. A drug-delivery device according to clause 14, in the form of an inhaler, comprising a mouthpiece coupled to the cyclonic-apparatus outlet.
16. A drug-delivery device according to clause 15, in the form of a dry-powder inhaler for delivering a dose of a medicament having an active pharmaceutical component wherein, in use, the cyclonic apparatus amplifies the pressure reduction caused by a user inhaling through the mouthpiece, and applies the amplified reduced pressure to the dose to release the active pharmaceutical component and enable that component to be inhaled through the mouthpiece.

1. A cyclonic apparatus having an inlet and an outlet and comprising:
a first cyclone chamber having a first cyclone inlet, a first reduced-pressure inlet, the first reduced-pressure inlet forming or in fluid connection with the cyclonic-apparatus inlet, and a first outlet forming the cyclonic-apparatus outlet;
the first cyclone chamber being operable to establish a cyclonic flow of fluid between the first cyclone inlet and the first outlet in response to fluid being drawn from the first outlet, so as to create a first reduced-pressure zone of fluid at the first reduced-pressure inlet,
in which the first reduced-pressure inlet is at an axial position downstream of the first cyclone inlet in the first cyclone chamber.

2. A cyclonic apparatus according to claim 1, comprising:
one or more further cyclone chambers arranged in series upstream of the first cyclone chamber, each further cyclone chamber having a cyclone inlet, a reduced-pressure inlet, and an outlet coupled to the reduced-pressure inlet of the next-downstream cyclone chamber;
each further cyclone chamber being operable to establish a cyclonic flow of fluid between its cyclone inlet and its outlet and to create a reduced-pressure zone of fluid at its inlet in response to fluid being drawn from its outlet by the reduced-pressure zone of fluid in the next-downstream cyclone chamber;
in which the pressure in the reduced-pressure zone of each cyclone chamber is lower than the pressure in the reduced-pressure zone of the next-downstream cyclone chamber;
and in which the reduced-pressure inlet of the cyclone at an upstream end of the series of cyclones forms the cyclonic-apparatus inlet.

3. A cyclonic apparatus according to claim 1, further comprising:
a second cyclone chamber having a second outlet coupled to the first reduced-pressure inlet,
a second cyclone inlet and a second reduced-pressure inlet,
the second chamber being operable to create a second reduced-pressure zone of fluid in response to fluid being drawn from the second outlet by the first reduced-pressure zone,
wherein the fluid in the second reduced-pressure zone is at a lower pressure than the fluid in the first reduced-pressure zone.

4. A cyclonic apparatus according to claim 3, further comprising:
a third cyclone chamber having a third outlet coupled to the second reduced-pressure inlet, a third cyclone inlet and a third reduced-pressure inlet,
the third chamber being operable to create a third reduced-pressure zone of fluid in response to fluid being drawn from the third outlet by the second reduced-pressure zone, wherein the fluid in the third reduced-pressure zone is at a lower pressure than the fluid in the second reduced-pressure zone.

5. A cyclonic apparatus according to claim 2, in which each cyclone chamber in the series progressively amplifies, at its reduced-pressure inlet, a reduced pressure applied, in use, to the cyclonic-apparatus outlet.

6. A cyclonic apparatus according to claim 1, in which each cyclone chamber is unidirectional.

7. A cyclonic apparatus according to claim 2, in which the cyclones are arranged coaxially with each other.

8. A cyclonic apparatus according to claim 1, in which the first cyclone chamber comprises a conduit having an inlet end and an outlet end, and being tapered from a larger diameter at the inlet end to a smaller diameter at the outlet end.

9. A cyclonic apparatus according to claim 2, in which each cyclone chamber comprises a conduit having an inlet end and an outlet end, and being tapered from a larger diameter at the inlet end to a smaller diameter at the outlet end.

10. A cyclonic apparatus according to claim 9, in which the conduits are coaxially arranged and in which the outlet end of the each conduit is nested or arranged within the inlet end of the next-downstream conduit.

11. A cyclonic apparatus according to claim 1, in which the cyclone inlet of the or each cyclone chamber is spaced along an axis of the cyclone formed, in use, in that cyclone chamber.

12. A cyclonic apparatus according to claim 11, in which the cyclone inlet of the or each cyclone chamber is tangential to the cyclone formed, in use, in that cyclone chamber.

13. A cyclonic apparatus according to claim 1, comprising two or more cyclone chambers arranged in series, in which each cyclone chamber has a higher airflow resistance than the next-downstream cyclone chamber.

14. A drug-delivery device in which a source of a medicament is coupled to the cyclonic-apparatus inlet of a cyclonic apparatus as defined in claim 1.

15. A drug-delivery device according to claim 14, in the form of an inhaler, comprising a mouthpiece coupled to the cyclonic-apparatus outlet.

16. A drug-delivery device according to claim 15, in the form of a dry-powder inhaler for delivering a dose of a medicament having an active pharmaceutical component wherein, in use, the cyclonic apparatus amplifies the pressure reduction caused by a user inhaling through the mouthpiece, and applies the amplified reduced pressure to the dose to release the active pharmaceutical component and enable that component to be inhaled through the mouthpiece.

17. A cyclonic apparatus according to claim 1, in which the cyclonic apparatus inlet is in fluid contact with an inhaler deagglomeration engine, such that the first reduced-pressure zone of fluid at the first reduced-pressure inlet drives the deagglomeration engine.

18. A cyclonic apparatus according to claim 3, in which each cyclone chamber in the series progressively amplifies, at its reduced-pressure inlet, a reduced pressure applied, in use, to the cyclonic-apparatus outlet.

19. A cyclonic apparatus according to claim 3, in which the cyclones are arranged coaxially with each other.

20. A cyclonic apparatus according to claim 3, in which each cyclone chamber comprises a conduit having an inlet end and an outlet end, and being tapered from a larger diameter at the inlet end to a smaller diameter at the outlet end.
